(19) United States
(12) Patent Application Publication
(10) Pub. No.: US 2003/0211516 A1
(43) Pub. Date: Nov. 13, 2003

(54) Compounds and methods for fluorescently labeling nucleic acids

(76) Inventor: Jonathan H Davis, Auburndale, MA (US)

Correspondence Address: CLARK & ELBING LLP, 101 FEDERAL STREET, BOSTON, MA, 02110, US

(21) Appl. No.: 10/312,264

(22) PCT Filed: Jul. 5, 2001
(86) PCT No.: PCT/US01/21304

(51) Int. Cl. C12Q 1/68

(52) U.S. Cl. 435/6

(57) ABSTRACT

The invention provides methods for the selection of nucleic acids that bind and thereby modulate (i.e, increase or decrease) the fluorescence intensity of a fluorophore. These selected nucleic acids may be used in methods, for example, for the detection, visualization, or quantitation of nucleic acids of interest.

### BACKGROUND OF THE INVENTION

In general, the invention features compounds and methods for monitoring gene expression and localization of encoded transcripts in vivo.

Monitoring gene expression is useful in the determination of factors, such as environmental conditions, that affect the expression of RNA molecules. In addition, determining the localization of RNA molecules or the interactions between these molecules may provide insights into the roles of these molecules in vivo.

Methods currently exist that allow protein expression to be imaged in living cells using green fluorescent protein (GFP), β-lactamase, or luciferase. For example, a protein of interest can be expressed as a fusion protein containing GFP, and fluorescence microscopy can be used to detect the intrinsic fluorescence of GFP, and thus determine the location of the fusion protein in a cell. Alternatively, the protein may be fused to β-lactamase, and the presence of the fusion protein may be detected based on the ability of β-lactamase to cleave a substrate and thus alter its fluorescence. Unfortunately, because the cleaved substrate can migrate within the cell, the location of the fluorescent signal from the substrate may not correspond to the location of the fusion protein, and the accumulation of cleaved substrate may hinder the detection of a decrease in protein expression over time. For the detection of a luciferase fusion protein, a cofactor must be added, and the light output is dependent upon other parts of the cellular machinery, such as ATP.

In addition, the above-mentioned methods for the detection of a protein of interest do not determine the presence or location of the corresponding transcripts or genes. Particular sequences of RNA or DNA can be identified in fixed cells by in situ hybridization using fluorescently labeled oligonucleotides. For the detection of specific nucleic acids in living cells, a fluorophore can be covalently attached to an RNA having a sequence complementary to a target RNA and microinjected into cells. In addition, dyes have been identified that bind tagged RNA transcripts; however, the use of these dyes is limited by the high background fluorescence of the unbound dye. Thus, an improved method is needed for the in vivo labeling of specific nucleic acids.

### SUMMARY OF THE INVENTION

The purpose of the present invention is to allow the visualization of specific RNA or DNA molecules, preferably in living cells. The methods of the present invention allow the selection of RNA or DNA molecules which bind to a fluorophore and thereby increase or decrease its fluorescence or which bind a quencher. When a cell permeable fluorophore is used in the methods of the invention, the fluorophore may be incubated with a cell under conditions that allow migration of the fluorophore into the cell and binding of the fluorophore to a nucleic acid of interest that is tagged with the selected molecule. The fluorescence intensity of the bound fluorophore may be used to determine the presence, location, or amount of the tagged nucleic acid of interest.

Accordingly, in a first aspect, the invention provides a method of selecting an RNA molecule which binds a fluorophore and thereby increases or decreases the fluorescence intensity of the fluorophore. This method involves (a) providing a population of candidate RNA molecules, (b) selecting the candidate RNA molecules which bind the fluorophore, (c) contacting the candidate RNA molecules from step (b) with the fluorophore, and (d) selecting the candidate RNA molecules which bind the fluorophore and increase or decrease its fluorescence intensity. In one preferred embodiment of this aspect, step (c) involves incubating cells with the fluorophore, the cells containing or expressing one or more candidate RNA molecules which bind the fluorophore in step (b). In another preferred embodiment of this aspect, the cells are sorted based on fluorescence intensity, and the DNA sequences encoding the candidate RNA molecules or the candidate RNA molecules are then recovered from the sorted cells.

In a related aspect, the invention features a method of selecting a DNA molecule which binds a fluorophore and thereby increases or decreases the fluorescence intensity of the fluorophore. This method involves (a) providing a population of candidate DNA molecules, (b) selecting the candidate DNA molecules which bind the fluorophore, (c) contacting the candidate DNA molecules from step (b) with the fluorophore, and (d) selecting the candidate DNA molecules which bind the fluorophore and increase or decrease its fluorescence intensity. In a preferred embodiment, step (c) involves incubating cells with the fluorophore, the cells containing or expressing one or more candidate DNA molecules shown to bind the fluorophore in step (b). In another preferred embodiment, the cells are sorted based on fluorescence intensity, and the candidate DNA molecules are then recovered from the sorted cells.

The selected molecules, called aptamers, which bind and increase or decrease the fluorescence of a fluorophore may be used in a number of methods for visualizing or quantifying nucleic acids. Fusion RNA or DNA molecules including a molecule of interest covalently linked to an aptamer may be monitored within a cell or sample due to the change in fluorescent intensity of the fluorophore which binds the aptamer. For example, a fusion RNA or DNA molecule containing an aptamer that binds and thereby increases the fluorescence intensity of a fluorophore may be detected based on the resulting increased fluorescence of the bound fluorophore compared to the unbound fluorophore. Alternatively, a fusion RNA or DNA molecule containing an aptamer that binds and thereby decreases the fluorescence intensity of a fluorophore may be detected based on the resulting area of decreased fluorescence due to the fluorophore bound to the aptamer compared to areas containing only unbound fluorophore. These methods enable characterization of the expression, localization, stability, or interactions between these molecules of interest, as well as the determination of compounds, environmental conditions, or genetic factors that effect these characteristics.

In one such method, the invention features a technique for determining the presence, location, or quantity of an RNA molecule of interest in a cell or an in vitro sample. This method involves (a) expressing, in the cell or sample, a fusion RNA molecule including the RNA molecule of interest covalently linked to an RNA aptamer, (b) contacting the cell or sample with a fluorophore under conditions that allow the RNA aptamer to bind the fluorophore and thereby increase or decrease its fluorescence, and (c) visualizing the fluorescence of the fluorophore.

The invention also provides a method for determining the presence, location, or quantity of a DNA molecule of interest in a cell or an in vitro sample. This method involves (a) expressing, in the cell or the sample, a fusion DNA molecule including the DNA molecule of interest covalently linked to a DNA aptamer, (b) contacting the cell or the sample with a fluorophore under conditions that allow the DNA aptamer to bind the fluorophore and thereby increase or decrease its fluorescence, and (c) visualizing the fluorescence of the fluorophore.

In another aspect, the invention provides a method of determining whether a compound is capable of modulating the transcription of an RNA molecule of interest. This method involves (a) expressing, in a cell or an in vitro sample, a fusion RNA including an RNA molecule of interest covalently linked to an RNA aptamer, (b) contacting the cell or the sample with either the compound and the fluorophore or with the fluorophore alone under conditions that allow the aptamer to bind and thereby increase the fluorescence of the fluorophore, and (c) measuring the fluorescence intensity in the presence and absence of the compound. The compound is determined to modulate transcription if it effects a change in the fluorescence intensity. The compound is determined to be an inhibitor of transcription if it decreases the fluorescence intensity of the fluorophore. Alternatively, the compound is determined to be an inducer if it increases the fluorescence. In one preferred embodiment, the compound is a member of a library of at least 50 compounds, all of which are simultaneously contacted with the cell or the sample.

In related aspect, the invention provides another method of determining whether a compound is capable of modulating the transcription of an RNA molecule of interest. This method involves (a) expressing, in a cell or an in vitro sample, a fusion RNA including an RNA molecule of interest covalently linked to an RNA aptamer, (b) contacting the cell or the sample with either the compound and the fluorophore or with the fluorophore alone under conditions that allow the aptamer to bind and thereby decrease the fluorescence of the fluorophore, and (c) measuring the fluorescence intensity in the presence and absence of the compound. The compound is determined to modulate transcription if it effects a change in the fluorescence intensity. The compound is determined to be an inhibitor of transcription if it increases the fluorescence intensity of the fluorophore. Alternatively, the compound is determined to be an inducer if it decreases the fluorescence. In one preferred embodiment, the compound is a member of a library of at least 50 compounds, all of which are simultaneously contacted with the cell or the sample.

In another aspect, the invention features a technique for determining whether a compound modulates the half-life of an RNA of interest. This method involves (a) expressing, in a cell or an in vitro sample, a fusion RNA molecule including an RNA of interest covalently linked to an RNA aptamer, (b) contacting the cell or the sample with either the compound and the fluorophore or with the fluorophore alone under conditions that allow the aptamer to bind and thereby increase the fluorescence of the fluorophore, and (c) measuring the fluorescence intensity in the presence and absence of the compound. The compound is determined to modulate the half-life of the RNA if the compound effects a change in the fluorescence intensity. The compound is determined to decrease the half-life if it reduces the fluorescence intensity and increase the half-life if it increases the fluorescence intensity. In one preferred embodiment, the compound degrades, induces the degradation, or suppresses the degradation the RNA of interest. In another preferred embodiment, the compound is a member of a library of at least 50 compounds, all of which are simultaneously contacted with the cell or the sample.

In a related aspect, the invention features another method for determining whether a compound modulates the half-life of an RNA of interest. This method involves (a) expressing, in a cell or an in vitro sample, a fusion RNA molecule including an RNA of interest covalently linked to an RNA aptamer, (b) contacting the cell or the sample with either the compound and the fluorophore or with the fluorophore alone under conditions that allow the aptamer to bind and thereby decrease the fluorescence of the fluorophore, and (c) measuring the fluorescence intensity in the presence and absence of the compound. The compound is determined to modulate the half-life of the RNA if the compound effects a change in the fluorescence intensity. The compound is determined to decrease the half-life if it increases the fluorescence intensity and increase the half-life if it decreases the fluorescence intensity. In one preferred embodiment, the compound degrades, induces the degradation, or suppresses the degradation the RNA of interest. In another preferred embodiment, the compound is a member of a library of at least 50 compounds, all of which are simultaneously contacted with the cell or the sample.

In yet another aspect, the invention provides a method of determining whether an antisense RNA molecule binds a target RNA molecule in a cell or an in vitro sample. This method involves (a) expressing, in the cell or the sample, a fusion RNA molecule including the target RNA covalently linked to an RNA aptamer, (b) contacting the cell or the sample with either the antisense nucleic acid and the fluorophore or with the fluorophore alone under conditions that allow the aptamer to bind and thereby increase the fluorescence of the fluorophore, and (c) measuring the fluorescence intensity in the presence and absence of the antisense nucleic acid. The antisense nucleic acid is determined to bind the target RNA if it decreases the fluorescence intensity. One possible mechanism by which the antisense nucleic acid may cause this decrease in fluorescence intensity involves the binding of the antisense nucleic acid to the target RNA molecule in such a way that the fluorophore is sterically prevented from binding to the aptamer. Another possible mechanism is the induction of a conformational change in the fusion RNA molecule upon binding to the antisense nucleic acid that reduces the affinity of the aptamer for the fluorophore. In one preferred embodiment, the antisense nucleic acid is a member of a library of at least 50 nucleic acid molecules, all of which are simultaneously contacted with the cell or the sample.

In a related aspect, the invention provides another method of determining whether an antisense RNA molecule binds a target RNA molecule in a cell or an in vitro sample. This method involves (a) expressing, in the cell or the sample, a fusion RNA molecule including the target RNA covalently linked to an RNA aptamer, (b) contacting the cell or the sample with either the antisense nucleic acid and the fluorophore or with the fluorophore alone under conditions that allow the aptamer to bind and thereby decrease the fluorescence of the fluorophore, and (c) measuring the fluorescence intensity in the presence and absence of the antisense nucleic acid. The antisense nucleic acid is determined to bind the target RNA if it increases the fluorescence intensity. In one preferred embodiment, the antisense nucleic acid is a member of a library of at least 50 nucleic acid molecules, all of which are simultaneously contacted with the cell or the sample.

In a related aspect, the invention features another method of determining whether an antisense nucleic acid binds a target RNA in a cell or an in vitro sample. This method involves expressing, in the cell or the sample, a fusion RNA including the target RNA covalently linked to an RNA aptamer which binds a first fluorophore. The cell or the sample is either contacted with the first fluorophore and the antisense nucleic acid covalently linked to a second fluorophore, or the cell or sample is contacted with the first fluorophore alone. This contacting occurs under conditions that allow the RNA aptamer to bind the first fluorophore and increase its fluorescence intensity. The emission wavelength of the second fluorophore is different from that of the first fluorophore, and the emission wavelength of the second fluorophore induces the fluorescence of the first fluorophore. The fluorescence intensity of the first fluorophore is measured in the presence and absence of the antisense nucleic acid. The antisense nucleic acid is determined to bind the target RNA if the antisense nucleic acid increases the fluorescence intensity of the first fluorophore. The increase in the fluorescence intensity may be due to fluorescence energy transfer between the second fluorophore, which is covalently linked to the antisense nucleic acid, and the first fluorophore bound to the RNA aptamer in the RNA fusion. In one preferred embodiment, the antisense nucleic acid is a member of a library of at least 50 nucleic acid molecules, all of which are simultaneously contacted with the cell or the sample.

In another related aspect, the invention features yet another method of determining whether an antisense nucleic acid binds a target RNA in a cell or an in vitro sample. This method involves expressing, in the cell or the sample, a fusion RNA including the target RNA covalently linked to an RNA aptamer which binds a fluorophore. The cell or the sample is either. contacted with the fluorophore and the antisense nucleic acid covalently linked to a quencher, or the cell or sample is contacted with the fluorophore alone. This contacting occurs under conditions that allow the RNA aptamer to bind the fluorophore and increase its fluorescence intensity. The fluorescence intensity of the fluorophore is measured in the presence and absence of the antisense nucleic acid. The antisense nucleic acid is determined to bind the target RNA if the antisense nucleic acid decreases the fluorescence intensity of the fluorophore. In one preferred embodiment, the decrease in the fluorescence intensity results from the absorption of the emission wavelength of the fluorophore by the quencher covalently bound to the antisense nucleic acid. In one preferred embodiment, the antisense nucleic acid is a member of at least 50 nucleic acid molecules, all of which are simultaneously contacted with the cell or the sample. Preferred quenchers include QSY-7 made by Molecular Probes, 6-carboxytetramethylrhodamine, and 4-((4-dimethylamino)phenyl)azo)benzoic acid, succinimidyl ester.

In yet another related aspect, the invention features another method of determining whether an antisense nucleic acid binds a target RNA in a cell or an in vitro sample. This method involves expressing, in the cell or the sample, a fusion RNA including the target RNA covalently linked to a RNA aptamer which binds a first fluorophore. The cell or sample is either (i) contacted with the first fluorophore, the antisense nucleic acid covalently linked to a nucleic acid aptamer which binds a second fluorophore, and the second fluorophore or (ii) contacted with the first fluorophore alone. This contacting occurs under conditions that allow the RNA aptamer to bind the first fluorophore and increase its fluorescence intensity. The contacting also allows the nucleic acid aptamer to bind the second fluorophore and, preferably, increase its fluorescence intensity. The emission wavelength of the second fluorophore is different from that of the first fluorophore, and the emission wavelength of the second fluorophore induces the fluorescence of the first fluorophore. The fluorescence intensity of the first fluorophore is measured in the presence and absence of the antisense nucleic acid. Alternatively, it is contemplated that the fluorescence intensity in the presence of the first fluorophore, the antisense nucleic acid, and the second fluorophore is compared to the fluorescence intensity in the absence of the second fluorophore or compared to the fluorescence intensity in the absence of the second fluorophore and the antisense nucleic acid. The antisense nucleic acid is determined to bind the target RNA if the antisense nucleic acid increases the fluorescence intensity of the first fluorophore. The increase in the fluorescence intensity may be due to the induction of the fluorescence intensity of the first fluorophore bound to the RNA fusion by the second fluorophore bound to the nucleic acid aptamer connected to the antisense nucleic acid. In one preferred embodiment, the antisense nucleic acid is a member of a library of at least 50 nucleic acid molecules, all of which are simultaneously contacted with the cell or the sample. It is also contemplated that the binding of the nucleic acid aptamer to the second fluorophore may not increase its fluorescence intensity.

In still another related aspect, the invention features yet another method of determining whether an antisense nucleic acid binds a target RNA in a cell or an in vitro sample. This method involves expressing, in the cell or the sample, a fusion RNA including the target RNA covalently linked to an RNA aptamer which binds a fluorophore. The cell or sample is either (i) contacted with the fluorophore, the antisense nucleic acid covalently linked to a nucleic acid aptamer which binds a quencher, and the quencher, or (ii) contacted with the fluorophore alone. This contacting occurs under conditions that allow the RNA aptamer to bind to the fluorophore and increase its fluorescence intensity. The contacting also allows the nucleic acid aptamer to bind the quencher. The fluorescence intensity of the fluorophore is measured in the presence and absence of the antisense nucleic acid. Alternatively, it is contemplated that the fluorescence intensity in the presence of the first fluorophore, the antisense nucleic acid, and the quencher is compared to the fluorescence intensity in the absence of the quencher or compared to the fluorescence intensity in the absence of the quencher and the antisense nucleic acid. The antisense nucleic acid is determined to bind the target RNA if the antisense nucleic acid decreases the fluorescence intensity of the first fluorophore. In one preferred embodiment, the decrease in the fluorescence intensity results from the absorption of the emitted wavelength of the fluorophore by the quencher bound to the nucleic acid aptamer. In one preferred embodiment, the antisense nucleic acid is a member of a library of at least 50 nucleic acid molecules, all of which are simultaneously contacted with the cell or the sample. Preferred quenchers include QSY-7 made by Molecular Probes, 6-carboxytetramethylrhodamine, and 4-((4-dimethylamino)phenyl)azo)benzoic acid, succinimidyl ester.

In another aspect, the invention features a method of determining whether a first RNA of interest is co-localized with a second RNA of interest in a cell or an in vitro sample. In this method, a first fusion RNA including the first RNA covalently linked to an RNA aptamer and a second fusion RNA including the second RNA of interest covalently linked to an RNA aptamer are expressed in the cell or the sample. The aptamer in the first fusion molecule is able to bind a first fluorophore, and the aptamer in the second fusion molecule is able to bind a second fluorophore, the second fluorphore having an emission wavelength that is different from that of the first fluorphore. The cell or the sample is contacted with both fluorophores, and the fluorescence of each fluorophore is visualized. The first RNA of interest is determined to co-localize with the second RNA of interest if the fluorescence of the first fluorophore is detected proximal to the fluorescence of the second fluorophore. Preferably, the binding of the RNA aptamers to the fluorophores increases the fluorescence intensity of the fluorophores.

In a related aspect, the invention features another method of determining whether a first RNA of interest is co-localized with a second RNA of interest in a cell or an in vitro sample. In this method, a first fusion RNA including the first RNA covalently linked to an RNA aptamer and a second fusion RNA including the second RNA of interest covalently linked to an RNA aptamer are expressed in the cell or the sample. The aptamer in the first fusion molecule is able to bind a quencher that decreases the fluorescence of a fluorophore. The aptamer in the second fusion molecule is able to bind and, preferably, increase the fluorescence of the fluorophore. The cell or the sample is contacted with the quencher and the fluorophore, and the fluorescence of the fluorophore is visualized. The first RNA of interest is determined to co-localize with the second RNA of interest if it effects a decrease in the fluorescence of the fluorophore. Preferred quenchers include QSY-7 made by Molecular Probes, 6-carboxytetramethylrhodamine, and 4-((4-dimethylamino)phenyl)azo)benzoic acid, succinimidyl ester.

In a related aspect, the invention provides a method of determining whether an RNA of interest is co-localized with a protein of interest in a cell or an in vitro sample. This method includes (a) expressing, in the cell or the sample, a fusion RNA including the RNA of interest covalently linked to an RNA aptamer which binds a first fluorophore, (b) expressing, in the cell or the sample, a fusion protein including the protein of interest covalently linked to a detectable protein, (c) contacting the cell or the sample with the first fluorophore under conditions that allow the RNA aptamer to bind the first fluorophore and increase its fluorescence intensity, and (d) visualizing both the detectable protein and the fluorescence of the first fluorophore. The RNA of interest is determined to co-localize with the protein of interest if the fluorescence of the first fluorophore is detected proximal to the detectable protein. In one preferred embodiment, the detectable protein has intrinsic fluorescence or luminescence, and the localization of the detectable protein is determined by visualizing its fluorescence or luminescence. Preferably, the detectable protein with intrinsic fluorescence is a green fluorescent protein. In another preferred embodiment, the method also involves contacting the cell or the sample with a second fluorophore under conditions that allow the detectable protein to bind the second fluorophore and, preferably, increase its fluorescence intensity; the localization of the detectable protein is determined by visualizing the fluorescence of the second fluorophore.

In a related aspect, the invention provides another method of determining whether an RNA of interest is co-localized with a protein of interest in a cell or an in vitro sample. This method includes (a) expressing, in the cell or the sample, a fusion RNA including the RNA of interest covalently linked to an RNA aptamer which binds a quencher, (b) expressing, in the cell or the sample, a fusion protein including the protein of interest covalently linked to a detectable protein, (c) contacting the cell or the sample with the quencher under conditions that allow the RNA aptamer to bind the quencher, and (d) visualizing the detectable protein. The RNA of interest is determined to co-localize with the protein of interest if it effects a decrease in the fluorescence of the detectable protein. In one preferred embodiment, the detectable protein has intrinsic fluorescence or luminescence, and the localization of the detectable protein is determined by visualizing its fluorescence or luminescence. Preferably, the detectable protein with intrinsic fluorescence is a green fluorescent protein. In another preferred embodiment, the method also involves contacting the cell or the sample with a fluorophore under conditions that allow the detectable protein to bind the fluorophore and, preferably, increase its fluorescence intensity; the localization of the detectable protein is determined by visualizing the fluorescence of the fluorophore. Preferred quenchers include QSY-7 made by Molecular Probes, 6-carboxytetramethylrhodamine, and 4-((4-dimethylamino)phenyl)azo)benzoic acid, succinimidyl ester.

In another aspect, the invention provides a method of determining whether a first RNA interacts with a second RNA in a cell or an in vitro sample. In this method, a first fusion RNA including the first RNA covalently linked to an RNA aptamer and a second fusion RNA including the second RNA of interest covalently linked to an RNA aptamer are expressed in the cell or the sample. The aptamer in the first fusion molecule is able to bind a first fluorophore, and the aptamer in the second fusion molecule is able to bind a second fluorophore. Additionally, the emission wavelength of the first fluorophore is different from that of the second fluorophore and induces the fluorescence of the second fluorophore. The cell or the sample is contacted with either both fluorophores or with the second fluorophore alone, and the fluorescence of the second fluorophore is visualized. The first RNA of interest is determined to interact with the second RNA of interest if the first fluorophore induces the fluorescence of the second fluorophore.

In another aspect, the invention provides a method of determining whether an RNA of interest interacts with a protein of interest in a cell or an in vitro sample. This method includes expressing, in the cell or the sample, a fusion RNA including the RNA of interest covalently linked to an RNA aptamer which binds a first fluorophore. Also expressed, in the cell or the sample, is a fusion protein including the protein of interest covalently linked to a detectable protein which binds a second fluorophore. The emission wavelength of the first fluorophore is different from that of the second fluorophore. Additionally, the emission wavelength of the first fluorophore induces the fluorescence of the second fluorophore, or the emission wavelength of the second fluorophore induces the fluorescence of the first fluorophore. The cell or the sample is contacted with either the first fluorophore and the second fluorophore, the first fluorophore alone, or the second fluorophore alone, under conditions that allow the RNA aptamer to bind the first fluorophore and increase its fluorescence intensity and that allow the detectable protein to bind the second fluorophore and, preferably, increase its fluorescence intensity. The fluorescence intensity of the first fluorophore is measured in the presence and absence of the second fluorophore, or the fluorescence intensity of the second fluorophore is measured in the presence and absence of the first fluorophore. The RNA of interest is determined to interact with the protein of interest if fluorescence resonance energy transfer occurs between the first fluorophore and the second fluorophore. It is also contemplated that the binding of the detectable protein to the second fluorophore may not increase its fluorescence intensity. Additionally, the method may be modified to use a detectable protein that binds or is covalently linked to a quencher that reduces the fluorescence of the first fluorophore instead of a detectable protein that binds a second fluorophore. Preferred quenchers include QSY-7 made by Molecular Probes, 6-carboxytetramethylrhodamine, and 4-((4-dimethylamino)phenyl)azo)benzoic acid, succinimidyl ester.

In a related aspect, the invention provides a method of determining whether an RNA of interest interacts with a protein of interest in a cell or an in vitro sample. This method involves expressing, in the cell or the sample, a fusion RNA including the RNA of interest covalently linked to an RNA aptamer which binds a fluorophore. A fusion protein including the protein of interest covalently linked to a detectable protein with intrinsic fluorescence is also expressed in the cell or the sample. The emission wavelength of the fluorophore is different from that of the detectable protein. Moreover, the emission wavelength of the fluorophore induces the fluorescence of the detectable protein, or the emission wavelength of the detectable protein induces the fluorescence of the fluorophore. The cell or the sample is contacted with the fluorophore under conditions that allow the RNA aptamer to bind the fluorophore and increase its fluorescence intensity. The fluorescence intensity of the fluorophore is measured in the presence and absence of the detectable protein, or the fluorescence intensity of the detectable protein is measured in the presence and absence of the fluorophore. The RNA of interest is determined to interact with the protein of interest if fluorescence resonance energy transfer occurs between the fluorophore and the detectable protein.

The invention also features populations of nucleic acid molecules that have particular secondary structures, such as those illustrated in FIGS. 1A-1C. These populations of nucleic acids may be used in a variety of selection methods to isolate nucleic acids that have a desired property, such as a desired activity or a desired binding affinity. For example, populations of candidate DNA or RNA molecules may be used in the methods of the present invention to select DNA or RNA molecules that bind and thereby modulate the fluorescence of a fluorophore.

In one such aspect, the invention features a population of nucleic acid molecules that have a loop flanked by a base-paired helix or that have two loops which are separated by a base-paired helix. The two loops may be the same or different sizes. In addition, the size of the loops may be the same or may vary between different nucleic acid molecules. In preferred embodiments, each side of the base-paired helix has between 1 and 10 nucleotides. The base-paired helix may contain nucleotides that all participate in Watson-Crick base-pairing or may contain one or more nucleotides that do not participate in Watson-Crick base-pairing. Preferred loops contain between 2 and 50 nucleotides or between 50 and 100 nucleotides. The nucleic acids in this population may also have other secondary structures, such as other loops, base-paired helices, or pseudoknots.

In a related aspect, the invention features a population of nucleic acid molecules that have a loop flanked by a base-paired helix on one side and a hairpin on the other side. In preferred embodiments, each side of the base-paired helix has between 1 and 10 nucleotides. The base-paired helix may contain nucleotides that all participate in Watson-Crick base-pairing or may contain one or more nucleotides that do not participate in Watson-Crick base-pairing. Preferred loops contain between 2 and 50 nucleotides or between 50 and 100 nucleotides. The two regions of the nucleic acids that form the loop may be the same length or may differ in length by one or more nucleotides (e.g., 3, 8, 17, or 39 nucleotides). Preferred hairpins contain a central UUCG tetraloop held together by a G-C or C-G basepair followed by several more basepairs, such as an additional four basepairs. Other preferred hairpins contain any stable tetraloop, such as GNRA, or any loop containing between 2 and 50 nucleotides. Some or all of the nucleotides in the loop of the hairpin may be randomized. Preferably, the hairpin contains a base-paired region which includes between 1 and 10 base-pairs. The nucleic acids in this population may also have other secondary structures, such as other loops, base-paired helices, or pseudoknots.

In still another aspect, the invention features a population of nucleic acid molecules that have a pseudoknot structure. The two regions of base-pairing in the pseudoknot structure may contain nucleotides that all participate in Watson-Crick base-pairing or may contain one or more nucleotides that do not participate in Watson-Crick base-pairing. Preferably, each side of a base-paired helix has between 3 and 10 nucleotides. For example, a base-paired helix may contain 6 basepairs. Preferably, the regions of the pseudoknot that are between the base-paired helices contain between 1 and 10 nucleotides, 10 and 20 nucleotides, or 20 and 30 nucleotides. The regions between the helices may be the same length or may differ in length by one or more nucleotides. The nucleic acids in this population may also have other secondary structures, such as other loops, base-paired helices, or pseudoknots.

In preferred embodiments of various aspects of the invention, the cell is a prokaryotic cell, such as a gram-negative or gram-positive bacterial cell. In other preferred embodiments, the cell is a eukaryotic cell. For example, the cell may be a yeast, Caenorhabditis, Xenopus, Drosophila, zebrafish, squid, plant, mammalian, embryonic, or human cell. Preferred zebrafish include Danio rerio, and preferred squid include Loligo pealei. In yet other preferred embodiments, the cell or the sample is contacted with the fluorophore by incubating the cell or the sample with the fluorophore. In still other preferred embodiments, the fluorophore is injected into the cell or administered to a plant, embryo, mammal, transgenic animal, or human including the cell.

In other preferred embodiments, the population of nucleic acids contains more than one DNA molecule or more than one RNA molecule. The nucleic acids may have naturally-occurring or non-naturally-occurring polynucleotide sequences. Preferably, regions of the nucleic acids; such as all or part of a loop, tetraloop, or helix; contain random sequences that differ between some or all of the members of the population. In other preferred embodiments, the sequence of a loop, tetraloop, or helix is the same in all of the members of the population. The lengths of any of the loops or helices may be the same or may differ between members of the population. The populations of nucleic acids may contain any number of unique molecules. For example, the population may contain as few as 10, 10², 10⁹, or 10¹¹ unique molecules or as many as 10³, 10¹⁴, 10¹⁵ or more unique molecules. In preferred embodiments, at least one of the polynucleotide sequences is a non-naturally-occurring sequence. Preferably, at least 10, 20, 40, 60, 80, 90, 95, 98, or 100% of the unique polynucleotide sequences are non-naturally-occurring sequences. The nucleic acids may either all have the same length or some of the molecules may differ in length. Preferably, the nucleic acids contain at least 10, 20, 40, 60, 80, 100, or 150 bases.

By “fluorophore” is meant a compound that is capable of emitting a fluorescent signal. As described herein, fluorophores of use in the invention have a higher fluorescence intensity when bound to a nucleic acid or protein than when unbound in solution. The fluorescence intensity of the bound fluorophore is preferably at least 5, 10, 50, 100, 500, or 1000 times that of the unbound fluorophore in an aqueous solution. Examples of conditions that may enhance the fluorescence of the bound fluorophore include rigidification, conformational restriction, and sequestration from solvent. In one preferred embodiment, the fluorophore does not covalently bind the aptamer.

Other preferred fluorophores have a lower fluorescence intensity when bound to a nucleic acid or protein than when unbound in solution. The fluorescence intensity of the bound fluorophore is preferably at least 2, 5, 10, 50, 100, 500, or 1000 less than that of the unbound fluorophore in an aqueous solution. An example of a condition that may decrease the fluorescence of the bound fluorophore is a change in the conformation of the fluorophore that decreases its fluorescence intensity. In one preferred embodiment, the fluorophore does not covalently bind the aptamer.

Preferably, the fluorophore does not substantially bind non-target molecules, which include other molecules in a cell or sample besides those molecules having a polynucleotide or amino acid sequence that was selected for binding to the fluorophore or having a sequence that is at least 70%, preferably 80%, or more preferably 90% identical to a selected sequence. For fluorophores that have increased fluorescence when bound to an aptamer of the invention, the fluorescence intensity of the fluorophore bound to these non-target molecules, as measured using the assays described herein, is 2-fold, preferably 5-fold, more preferably 10-fold, and most preferably 50-fold lower than that of the fluorophore bound to molecules having a selected sequence or a sequence that is at least 70%, preferably 80%, or more preferably 90% identical to a selected sequence. The lower fluorescence intensity of the fluorophore bound to non-target molecules may be due to a smaller number of fluorophore molecules bound to the non-target molecules or a reduced enhancement of fluorescence intensity upon binding the non-target molecules compared to binding to molecules having a selected sequence. For fluorophores that have decreased fluorescence when bound to an aptamer of the invention, the fluorescence intensity of the fluorophore bound to these non-target molecules, as measured using the assays described herein, is 2-fold, preferably 5-fold, more preferably 10-fold, and most preferably 50-fold greater than that of the fluorophore bound to molecules having a selected sequence or a sequence that is at least 70%, preferably 80%, or more preferably 90% identical to a selected sequence. The greater fluorescence intensity of the fluorophore bound to non-target molecules may be due a reduced inhibition of fluorescence intensity upon binding the non-target molecules compared to binding to molecules having a selected sequence.

Other preferred fluorophores, such as calcium-sensing dyes, may adopt at least two different conformational states that result in different fluorescence intensities. An aptamer of the invention may modulate the fluorescence of the fluorophore by increasing the percentage of the fluorophore in a particular conformational state with increased or decreased fluorescence.

Preferably, the fluorophore is water soluble, non-toxic, and cell permeable; examples of such fluorophores include cyanine dyes. Preferably, the fluorophore is soluble in an aqueous solution at a concentration of 0.1 μM, 1 μM, more preferably 10 μM, and most preferably 50 μM. Preferably, incubating a cell with these concentrations of the fluorophore does not effect the viability of the cell. In another preferred embodiment, incubating a cell with the fluorophore at these concentrations for as few as 1 or 2 hours to as many as 8, 12, 24, 36, or more hours does not require the presence of another compound to prevent toxic effects of the fluorphore, such as the inactivation of proteins in the cell; inhibition of replication, transcription, or translation; or the induction of cell death. Preferably, the fluorophore does not contain arsenic. By “cell permeable” is meant capable of migrating through a cell membrane or cell wall into the cytoplasm or periplasm of a cell by active or passive diffusion. Preferably, the fluorophore is able to migrate through both the outer and inner membranes of gram-negative bacteria or both the cell wall and plasma membrane of plant cells. Additionally, the fluorophore may be used to visualize nucleic acids in an in vitro sample.

By “DNA” is meant a sequence of two or more covalently bonded naturally-occurring or modified deoxyribonucleotides.

By “RNA” is meant a sequence of two or more covalently bonded naturally-occurring or modified ribonucleotides. Examples of RNA molecules include mRNA, tRNA, and sRNA molecules.

By “covalently linked” is meant covalently bonded or connected through a series of covalent bonds.

By “aptamer” is meant a molecule that binds a second molecule. As used herein, preferred aptamers bind and thereby increase the fluorescence intensity of a fluorophore or bind and thereby decrease the fluorescence intensity of a fluorophore. Other preferred aptamers bind a quencher, which is a molecule that reduces the fluorescence intensity of a fluorophore, possibly through absorbing the emitted wavelength of the fluorophore. These aptamers may be selected from pools of candidate molecules using the methods of the invention. For the selection of aptamers that bind a quencher, an in vitro selection may be performed to select candidate molecules that bind an immoblized quencher as described for the selection of aptamers that bind an immoblized fluorophore. Any quencher may be used in these methods.

By “selecting” is meant substantially partitioning a molecule from other molecules in a population. Preferably, the partitioning provides at least a 2-fold, preferably, a 30-fold, more preferably, a 100-fold, and most preferably, a 1,000-fold enrichment of a desired molecule relative to undesired molecules in a population following the selection step. The selection step may be repeated a number of times, and different types of selection steps may be combined in a given approach. The population preferably contains at least 10⁹ molecules, more preferably at least 10¹¹, 10¹³, or 10¹⁴ molecules and, most preferably, at least 10¹⁵ molecules.

By “contacting the cell with the fluorophore” is meant incubating the cell with a solution having the fluorophore, growing the cell on agar or other solid media having the fluorophore, applying the fluorophore or a solution having the fluorophore to the cells, or injecting the fluorophore into the cell. In other preferred embodiments, the fluorophore may be administered to a mammal, embryo, or plant having the cell. Preferred routes of administered to a mammal include oral and parenteral (i.e., intravenous, intramuscular, intravascular, subcutaneous, or intraperitoneal). A plant having the cell may also be contacted with the fluorophore such that the fluorophore is taken up into the plant, possibly by migration of a solution having the fluorophore into a root of the plant. It is also contemplated that the compounds and antisense molecules used in the methods of the invention may be expressed in the cell or the sample rather than added to the cell or the sample. For example, the compounds that modulate the transcription or half-life of an RNA of interest may include proteins or nucleic acids that are expressed in the cell or the sample. In a preferred embodiment, the antisense nucleic acids are antisense RNA molecules that are expressed in the cell or the sample.

By “increasing the fluorescence intensity” is meant enhancing the fluorescence intensity of a fluorophore such that the intensity is preferably 2, 5, 10, 50, 100, 500, or 1000 times that of the unbound fluorophore or the fluorophore in the absence of the nucleic acid that enhances its fluorescence. Preferably, the nucleic acid noncovalently binds the fluorophore through an interaction such as an ion-ion force, dipole-dipole force, hydrogen bond, van der Waals force, electrostatic interaction, or any combination of these interactions. It is also contemplated that the nucleic acid cleaves the fluorophore, induces the formation or breaking of a covalent bond between atoms of the fluorophore, otherwise alters the covalent structure of the fluorophore, or covalently binds the fluorophore resulting in a product with an increased fluorescence intensity.

By “decreasing the fluorescence intensity” is meant inhibiting the fluorescence of a fluorophore such that the fluorescence intensity is preferably 2, 5, 10, 50, 100, 500, or 1000 fold less than that of the unbound fluorophore or the fluorophore in the absence of the nucleic acid that decreases its fluorescence. Preferably, the nucleic acid noncovalently binds the fluorophore through an interaction such as an ion-ion force, dipole-dipole force, hydrogen bond, van der Waals force, electrostatic interaction, or any combination of these interactions. It is also contemplated that the nucleic acid cleaves the fluorophore, induces the formation or breaking of a covalent bond between atoms of the fluorophore, otherwise alters the covalent structure of the fluorophore, or covalently binds the fluorophore resulting in a product with a decreased fluorescence intensity.

By “effects a change in the fluorescence intensity” is meant causes a change in the fluorescence intensity. For example, the fluorescence intensity may change because of an alteration in the number or stability of molecules that are present in a cell or sample (and that are therefore available to bind and increase the fluorescence intensity of the fluorophore). Alternatively, a compound may effect a change in the number of bound fluorophore molecules. A fluorophore may increase the fluorescence intensity of another fluorophore through fluorescence resonance energy transfer, or a quencher may decrease the fluorescence intensity of a fluorophore through fluorescence quenching. Preferably, the change in fluorescence intensity is at least 2, 5, 10, 25, or 50 times the fluorescence intensity in the absence of the second fluorophore or in the absence of the quencher.

By “recovering” is meant isolating or identifying the polynucleotide sequence. For example, the sequence of a candidate DNA molecule in a cell may be determined using standard techniques, such as amplifying the DNA molecule using a polymerase chain reaction and sequencing the amplified DNA. The sequence of a candidate RNA molecule may be determined by reverse transcribing the RNA, optionally PCR amplifying, and sequencing the resulting cDNA.

By “visualizing” is meant detecting, localizing, or quantifying. The fluorescence of the fluorophore may be detected or quantitated using standard techniques, such as fluorescence microscopy, fluorimetry, or fluorescence-activated cell sorting (FACS) (see, for example, Stearns, Curr. Biol. 5(3):262-264, 1995; Imai et al., Exp. Nephrol. 7(1):63-66, 1999; Yang, et al. Gene 173:19-23, 1996; Chalfie et al., Science 263:802-805, 1994; Chalfie et al., The glow spreads throughout biology: Green Fluorescent Protein: Properties, Applications, and Protocols, Wylie-Liss, 1998).

By “detected proximal” is meant located within 1, preferably 0.1, more preferably 0.01, and most preferably, 0.001 microns.

By “induces the fluorescence” is meant causes an increase in fluorescence intensity by exciting a fluorophore with the emitted light of another fluorophore in a process called fluorescence resonance energy transfer (FRET).

The present invention provides a number of advantages related to the visualization of nucleic acids in cells or other samples. For example, the turnover of the molecules in a cell which bind the fluorophore is expected to minimize photo damage, which might otherwise occur during the imaging of cells for extended periods of time, and allow the detection of changes in the fluorescence intensity over time. In addition, because the methods of selecting a nucleic acid that binds and thereby increases or decreases the fluorescence of a fluorophore may be performed for a variety of fluorophores, a set of fluorophores with a range of emission characteristics may be used simultaneously for applications in which it is desirable to visualize more than one nucleic acid of interest. Moreover, the assays are simple to perform and amenable to high-throughput analysis.

Other features and advantages of the invention will be apparent from the following detailed description and from the claims.

### BRIEF DESCRIPTION OF THE DRAWING

FIGS. 1A-1C are a set of schematic illustrations of candidate DNA and RNA molecules used in the selection for nucleic acids that bind and thereby enhance or decrease the fluorescence intensity of a fluorophore or that bind a quencher. FIG. 1A is a schematic illustration of nucleic acids having a loop flanked by a base-paired helix or having two loops of various sizes that are separated by a base-paired helix. One of the loops is denoted R1. The other loop consists of regions R2 and R3. In some of the candidate molecules, regions R2 and/or R3 are absent. FIG. 1B is an illustration of nucleic acids having a loop flanked by a base-paired helix (e.g., a six-base-paired helix) on one side and a hairpin on the other side. The loop consists of regions R1 and R2 which may differ in length or which may be the same length. FIG. 1C is an illustration of nucleic acids having a pseudoknot structure. The regions of the pseudoknot that are between base-paired regions are donted Link, R1 and R2.

FIG. 2 is a list of sample templates and primers used for the synthesis of candidate RNA or DNA molecules. X designates a region of random nucleotides, and the subscript refers to the length of the random region. Ran-63 refers to a template with a random region that is 63 nucleotides long. L-58 refers to a template that has a loop with 58 nucleotides, as illustrated in FIG. 1A. PKL-3 refers to a template with a pseudoknot structure that has a link region with 3 nucleotides (FIG. 1C). BΔ0 refers to a template for a bulge aptamer in which the length of random regions R1 and R2 differs by 0 nucleotides (FIG. 1B). HP-UUCG refers to a hairpin template. This template has a CGAA sequence between the two random regions that is transcribed into an UUCG sequence known to form a stable tetraloop. HP-Ran6 has a random region of 6 nucleotides (X₆) designed to form a hexaloop that is flanked by a stem, this sternloop may interact with various structures formed from the random region. The stem is formed from the five nucleotides flanking the X₆ random region (i.e., GTGCA and GTACA).

Primer 1 binds to the 3′ end of RNA to initiate reverse transcription or to the 3′ end of the top DNA strands to initiate polymerase chain reaction (PCR). The top DNA strand is the strand that has a sequence complementary to and binds the template DNA strand. Primer 2 binds to the 3′ end of the templates. This primer may be used to begin synthesis of the top strands without the T7 promoter. In addition, radioactive phosphate may be added to the primer using a kinase, allowing the radioactive primer to be used in restriction analysis based on standard procedures. Primer 3 includes the top strand of the T7 promoter and binds to the 3′ end of the templates for use in PCR and T7 transcription. When this primer is used for PCR amplification of cDNA, formed from the reverse transcription of candidate RNA molecules, the resulting PCR product contains the T7 promoter which may be used to initiate transcription to form RNA. This primer may also be used for transcription of single stranded DNA.

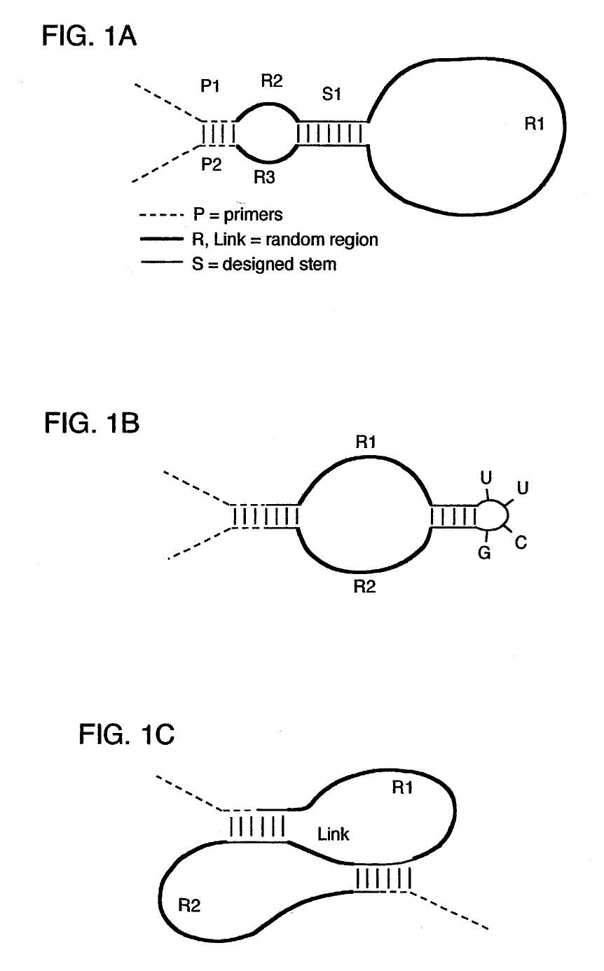

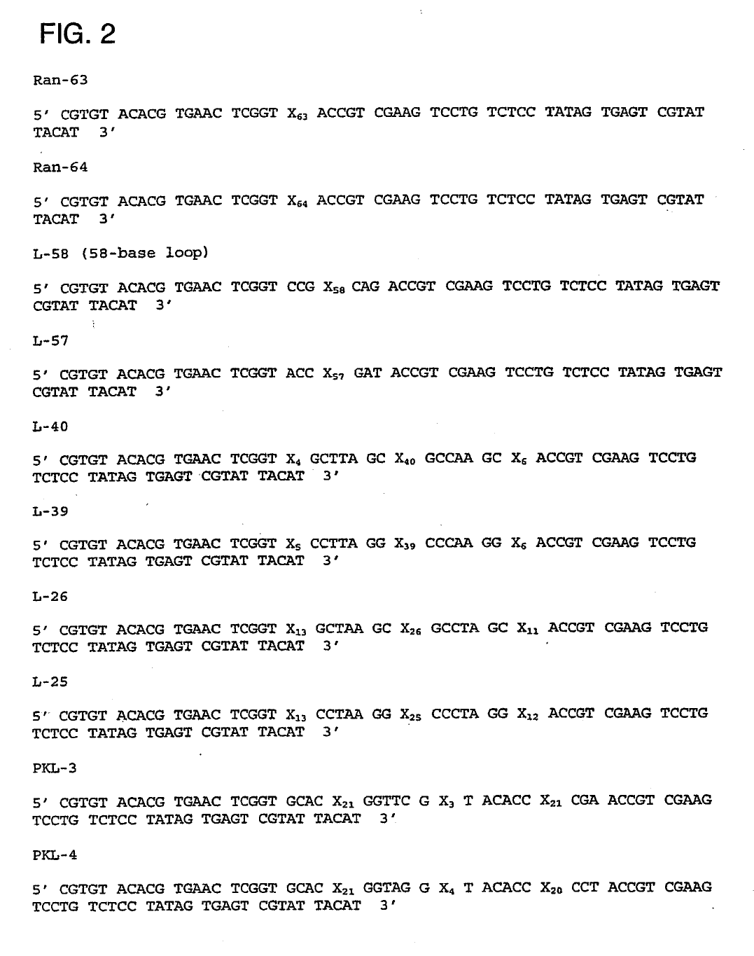

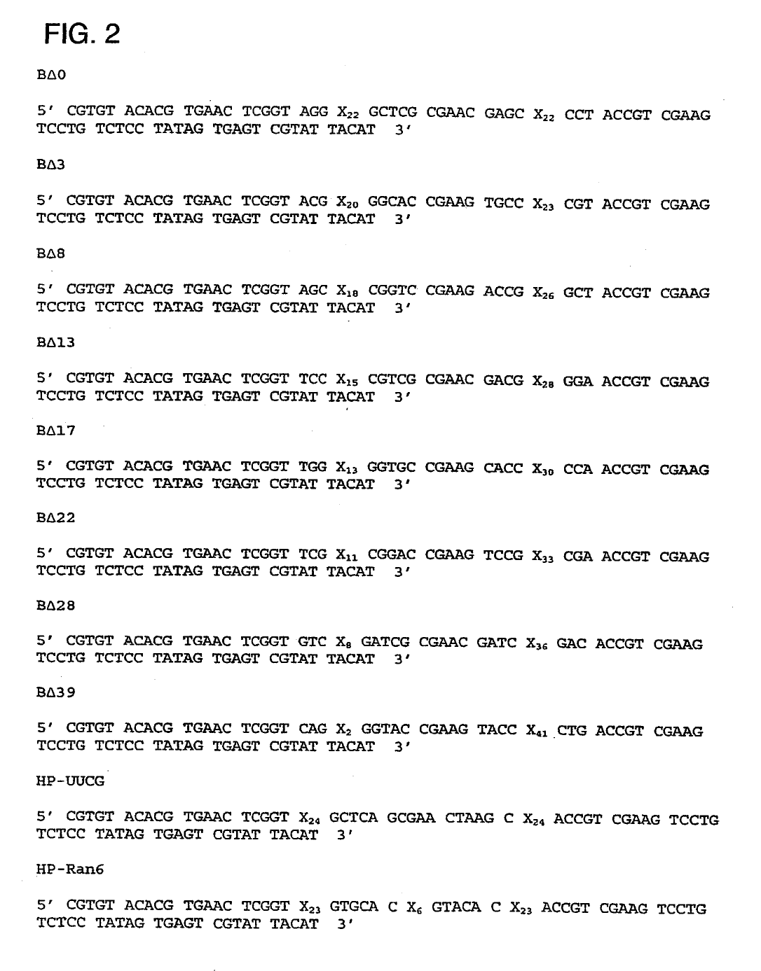

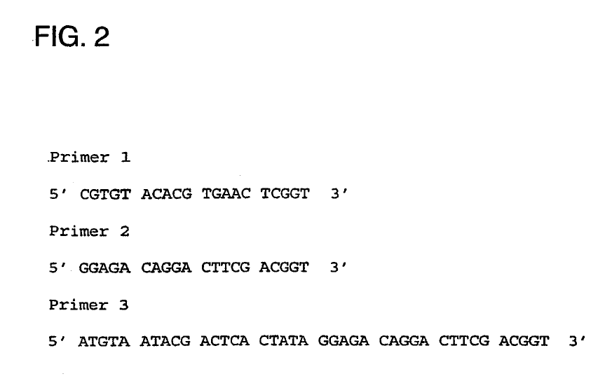

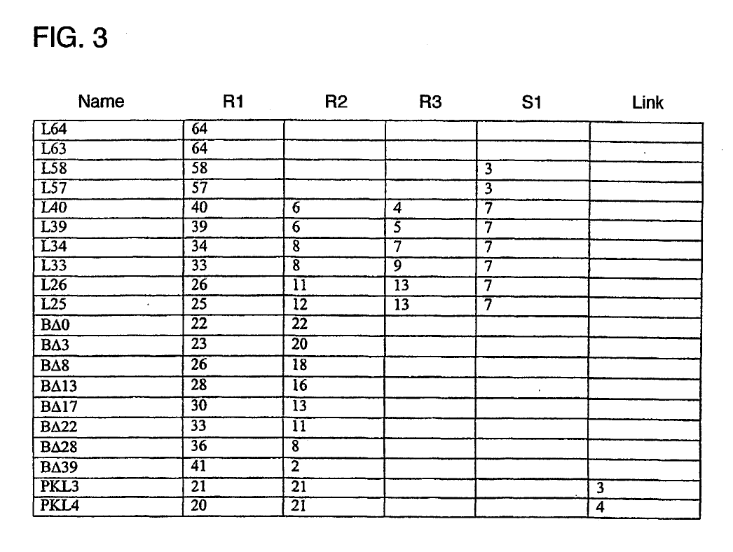

FIG. 3 is a table that lists the final lengths for the variable regions in the DNA templates described in Example 1. These templates were used for the synthesis of three sets of candidate RNA molecules that have the general structures described in FIGS. 1A-1C. Columns R1, R2, and R3 contain the lengths of the random regions in these molecules. The S1 column contains the length of the base paired helix that flanks a loop (denoted R1) or that separates two loops of various sizes in molecules having the structure illustrated in FIG. 1A. The Link column lists the length of the linker in the pseudoknot molecules described in FIG. 1C.

### DETAILED DESCRIPTION

We have developed methods for selecting RNA and DNA aptamers that bind and modulate (i.e., increase or decrease) the fluorescence of a fluorophore. These aptamers may be used to construct gene fusions for the expression of fusion RNA or DNA molecules including an aptamer and a molecule of interest. The invention also provides sensitive methods for the visualization of these fusion molecules. These methods have a broad range of applications, including those that require the determination of the presence, localization, migration, or quantity of a specific nucleic acid in a living cell. In addition, these methods enable the determination of the time at which a nucleic acid is formed or degraded in the cell, as well as the identification of compounds or environmental conditions that affect this formation or degradation. These methods may also be used to detect the co-localization of or interactions between nucleic acids. Moreover, methods are provided for identifying compounds that effect the co-localization of or interactions between these molecules.

The invention also features a method to identify RNA molecules that are present in increased or decreased levels or in certain locations under various conditions or at different stages of the cell-cycle. For example, DNA encoding an RNA aptamer can be added in directed or random insertions to the DNA of a cell, and the cell can be incubated in buffer containing the fluorophore. The cell can then be visualized under various conditions over time to monitor the expression of RNA molecules in which the aptamer is present. RNA with the desired expression patterns may be identified by isolating the DNA from the cell and amplifying the DNA encoding the RNA of interest using primers designed to bind the DNA sequence of the aptamer.

Because these methods may be used to better understand the cellular processes of normal, diseased, or recombinant cells, these methods are relevant to studies of a variety of diseases, disorders, and infections.

The following examples are provided to illustrate the invention. They are not meant to limit the invention in any way.

### EXAMPLE 1

### Selection of DNA Molecules that Bind and Thereby Increase the Fluorescence Intensity of a Fluorophore

Design of Candidate DNA Molecules

To design candidate DNA molecules, pools of candidate DNAs are generated, for example, by the method of U.S. Pat. No. 5,270,163 or Ellington et al. (Nature 346:818-822, 1990). The pools may include molecules of various lengths. The candidate molecules may either all have the same length or some of the molecules may differ in length. Preferably, the molecules contain at least 10, 20, 40, 60, 80, 100, or 150 bases. The number of candidate molecules in the candidate population may also vary. For example, the population may contain as few as 10², 10⁹, or 10¹¹ molecules or as many as 10¹³, 10¹⁴, 10¹⁵ or more molecules. The candidate DNA molecules may have one or more regions containing a random sequence of bases. The candidate molecules may also contain one or more sequences designed based on common motifs in nucleic acids. For example, the designed DNA sequences may correspond to nucleic acid motifs that have been selected to bind a small molecule. The motifs may contain various numbers of bases, such as at least 5, 10, 20, 40, or 60 bases. Possible motifs include loops, loops flanked by base-paired helix, hairpins, hairpins plus an outer stem, pseudoknots, any other secondary structure formed by nucleic acids, or a combination of these motifs. Examples of some of the possible motifs are illustrated in FIGS. 1 and 3. The presence of defined structures in the candidate molecules is expected to allow more of the possible sequences in the random regions to be present in the original pool.

For this example, a pool of about 10¹⁶ unique DNA molecules was synthesized (U.S. Pat. No. 5,270,163 and Ellington et al., supra) with 63 or 64 bases between the two fixed primers to maximize the chance of finding a DNA molecule that folds into a structure that enables it to bind and increase the fluorescence of a fluorophore. The candidate DNA molecules consist both of molecules that have random sequences of bases between the primers and molecules having random sequences plus sequences designed based on common motifs found in RNA sequences that have been selected to bind a small molecule. Twenty pools of candidate DNA molecules were designed and synthesized, which include random pools; loops of varying sizes flanked by a base-paired helix (FIG. 1A); a hairpin centered in a random region (FIG. 1B); a hairpin plus an outer stem defining 12 of the possible bulge sizes (FIG. 1B); and two pseudoknot skeletons with random loop regions (FIG. 1C).

For candidate molecules with a loop flanked by a strong base-paired helix, the helix (S1 in FIG. 1A) is expected to provide rigidity and closure to the molecules which may enhance binding to the fluorophore. A set of candidate molecules have been designed and synthesized with the following lengths for the R1, R2, and R3 random regions and for each side of the S1 base-paired stem, as illustrated in FIG. 1A. For L64 (i.e., a nucleic acid with a loop of length 64), R1 is 64 bases long; S1, R2, and R3 are absent. For L63, R1 is 63 bases long; S1, R2, and R3 are absent. In L58, R1 has 58 bases, and S1 has 3; R2 and R3 are absent. For L57, R1 contains 57 bases, and S1 contains 3; R2 and R3 are absent. For L40, R1 is 40; S1 is 7; R2 is 6; and R3 is 4 bases long. For L39, R1 has 39 bases; S1 has 7; R2 has 6, and R1 has 5 bases. For L34, R1 has 34 bases; S1 has 7 bases; R2 has 8; and R3 has 7 bases. For L33, R1 has 33 bases; S1 has 7 bases; R2 has 8; and R3 has 9 bases. For L26, R1 is 26 bases long; S1 is 7; R2 is 11; and R3 is 13 bases long. In L25, R1 is 25; S1 is 7; R2 is 12; and R3 is 13 bases long. The lengths of the random regions and stems were chosen to sample the range of possible nucleic acids. DNA sequences with loops or stems of other lengths may also be used. It is also contemplated that the nucleotides in the R1 region that flank the S1 stem may base-pair resulting in the elongation of the S1 stem and the shortening of the loop containing the remaining R1 sequence. Each base-pair that is formed shortens the loop by two bases, further diversifying the sizes of the loops in the candidate molecules.

The sequences of the stem regions for molecules with different sets of R1, R2, and R3 lengths were designed to differ by at least one base pair to minimize heterodimerization between the stem regions of different molecules and to generate different restriction sites within the stems. The presence of different restriction sites in molecules with different sets of R1, R2, and R3 lengths may be used to rapidly identify the different sets of molecules based on the size of the restriction enzyme-digested products and the susceptibility to cleavage by different restriction enzymes. This information may be used to determine which sets of molecules are the most abundant after a particular round of selection. The sequence for each of the two primers is the same for all the candidate molecules and was designed to form a 4-base-paired helix next to the R2 and R3 regions which may further stabilize the closure of the R1 loop. The primers consist of 20 nucleotides with a GC content of approximately 55%; however, other lengths and sequences may be used.

For candidate molecules with a loop flanked by a base-paired helix and a hairpin, the following nucleic acid sequences have been synthesized with random regions R1 and R2, as illustrated in FIG. 1B. For BΔ0 (i.e., a sequence in which the lengths of R1 and R2 differ by 0 bases), R1 and R2 have 22 bases. In BΔ3, R1 is 23, and R2 is 20 bases long. For BΔ8, R1 is 26, and R2 is 18 bases. For BΔ13, R1 is 28, and R2 is 16 bases. In BΔ17, R1 is 30, and R2 is 13 bases. For BΔ22, R1 contains 33, and R2 contains 11 bases. In BΔ28, R1 is 36, and R2 is 8 bases in length. For BΔ39, R1 has 41, and R2 has 2 bases. The lengths of the random regions were chosen to sample the possible range of nucleic acids; however, other lengths may be used. The sequence of the hairpin contains a central UUCG tetraloop held together by a G-C or C-G basepair followed by several more basepairs, usually an additional four basepairs. This sequence is known to form a stable hairpin, and thus, may nucleate the folding of the candidate molecules into their secondary structures. The stem, which is on the opposite side of the R1 and R2 regions as the hairpin, contains 7 bases-pairs with different restriction sites for each set of. molecules with a particular set of R1 and R2 lengths. Candidate molecules were also made in which the three basepairs from this 7 base-pair helix that are proximal to the random regions were replaced by random sequences.

Candidate nucleic acids have also been designed to have a pseudoknot structure, as illustrated in FIG. 1C. The sequence PKL3 has a length of 21 bases for R1, 21 bases for R2, and 3 bases for the Link region. For the PKL4 sequence, R1 has 20 random bases, R2 has 21, and the Link has 4. Other lengths may be used for these regions. The two regions of base-pairing illustrated by vertical lines in FIG. 1C each contain 6 base-pairs. Longer or shorter base-paired regions may also be used in the design of other candidate molecules.

Immobilization of Fluorophore

A variety of fluorophores may be used for the selection. Preferably, the fluorophore has a low fluorescence intensity when unbound in aqueous solution and a high fluorescence intensity when bound or otherwise sequestered from water. Possible fluorophores include dyes that do not bind nucleic acids but have a structure similar to a dye that nonspecifically binds nucleic acids, such as a nucleic acid stain. Other examples of possible candidate dye molecules include cyanine dyes or any dye that has a functional group, such as a hydroxyl, carboxyl, or amide, on an aromatic ring.

For immobilization, the fluorophore may be synthesized with a linker that is terminated by an activated group, such as a maleimide, isothiocyanate, succinate, or sulfhydyl group, that can be covalently attached to a solid support, such as a column matrix that has a proper accepting group, such as an amide or thiopyradone. Alternatively, readily available sepharose matrices may be utilized that have linkers terminated by activated groups, such as epoxides, that can be reacted directly with the fluorophore, provided the fluorophore contains the proper reactive group, such as an amide or hydroxyl group. Once the fluorophore is immobilized, the matrix containing the fluorophore may then be poured into a column.

Selection for molecules that bind to small molecules connected to columns in this manner can produce sequences that fold around the ligand, leaving the region connected to the linker exposed (Dieckmann et al., RNA 2:628, 1996 and Zimmermann et al., Nature Struct. Biol. 4:644, 1997). To increase the likelihood of selecting DNA molecules that not only bind the fluorophore but also increase its fluorescence intensity, possibly by sequestering the fluorophore from water, several different linker or connecting groups should be utilized to allow binding of the DNA molecules in several different orientations.

Selection of DNA Molecules that Bind the Fluorophore

The DNA molecules that bind the fluorophore may be selected using a modification of the Systematic Evolution of Ligands by Exponential Enrichment (SELEX) method (U.S. Pat. No. 5,270,163 and Ellington et al., Nature 346:818-822, 1990). The pool of candidate DNA molecules is added to a buffer that closely mimics intracellular conditions (15 mM NaCl, 100-240 mM KCl, 0.5-5.5 mM Mg₂Cl, buffered to pH 7.1 with 5 mM PO₄.). For early rounds of the selection, a higher concentration of KCl and Mg²⁺ is used (240 mM KCl and 5.5 mM Mg₂Cl) because salts, particulary Mg²⁺, can stabilize nucleic acid structures. For later rounds of selection, the concentration of KCl and Mg²⁺ may be lowered to more physiological concentrations (100 mM KCl and 0.5 mM Mg₂Cl).

The DNA is applied to the column containing the immobilized fluorophore. The column is washed to elute the non-binding and weakly-binding molecules from the column, and then the bound DNA molecules are specifically eluted by the addition of buffer containing the fluorophore. The DNA molecules can dissociate from the immobilized fluorophore and bind the fluorophore in solution instead of rebinding the immobilized fluorophore. The eluted DNA may be amplified using PCR for use in additional rounds of selection. Repeating the selection several times may enrich the population for DNA molecules that bind the fluorophore. It is expected that, after 4-8 rounds of selection, most of the remaining DNA molecules will be able to bind the fluorophore. The best binders can be cloned, sequenced, and characterized, using standard techniques (see, for example, Ausubel et al., Current Protocols in Molecular Biology, volume 2, p. 11.13.1-11.13.3, John Wiley & Sons).

Alterations to the above method may be performed to generate additional or higher affinity binders to the fluorophore. For example, mutagenic and/or recombinant PCR may be used in any round of the selection to generate variations of the selected DNA molecules. Using mutagenic PCR early in the selection, such as in rounds 3 or 4, may allow the resulting variations of the selected DNA molecules to become reasonably common before the in vivo selection is performed. Additionally, various regions in the candidate molecules that are isolated from a selection may be mutated to generate a library of candidate molecules to be used in a second selection. This second selection may further optimize the affinity of the candidate molecules for the fluorophore and may allow the regions that are important for binding (i.e., the regions in which binding is most affected by mutations) to be determined.

The stringency of the selective pressure may also be increased after the mutagentic PCR step, which preferably is conducted in round 3 of the selection. For example, less fluorophore can be added to the matrix, and a larger number of candidate DNA molecules can be used for the next round of selection or any subsequent rounds of selection. If the molarity of the DNA exceeds that of the immobilized fluorophore, then after incubating the DNA with the immobilized fluorophore, an equilibrium may be established, with the tighter binding DNA molecules preferentially bound to the column. To select for tight binding DNA molecules, the column may be washed extensively with buffer before specifically eluting the bound DNA molecules with the fluorophore. To select for DNA molecules that have a slow rate of dissociation from the fluorophore, the column may be washed with less buffer and then washed with several column-volumes of buffer containing the fluorophore. The buffer containing the fluorophore is then incubated for several minutes on the column. After one or more of these washes with the fluorophore in solution, the bound DNA molecules may be eluted by incubating for several hours with the buffer containing the fluorophore. The DNA molecules that elute last, and thus have the slowest off-rate, are collected.

Selection for DNA Molecules Which Bind and Increase the Fluorescence Intensity of the Fluorophore

The selected DNA molecules which bind the fluorophore are then further tested to determine which molecules also enhance the fluorescence of the fluorophore. For this selection, the DNA molecules are added to PCR reaction mixture such that only a few (1-10) different DNA molecules are present in each mixture. The DNA sequences are amplified as single stranded DNA by using either asymmetric PCR amplification (Gyllenstein et al., PNAS 85:7625-7656, 1988 and Ellington et al., Nature 355:850, 1992) or standard PCR using two primers followed by PCR using only one primer (Huizenga et al., Biochemistry 34:656, 1995). The amplification of the DNA may be performed in 96 well plates or the PCR products may be transferred to 96 well plates. The fluorophore is then added to the wells, and the plates are analyzed using a fluorescence imager.

To identify the DNA molecules in the wells with the highest fluorescence intensity, the DNA molecules from these wells are ligated to a restriction enzyme-digested plasmid using standard molecular biology techniques (Ausubel et al., supra). The ligated plamids are then transfected into bacteria, and the bacteria are grown as single colonies. The plasmids are isolated from several colonies, and the sequences of the candidate DNA molecules that were inserted into the plasmids are determined using DNA sequencing. The identified candidate DNA molecules are then tested individually to determine which of the several molecular species in the well were responsible for the fluorescence enhancement.

### EXAMPLE 2

### Selection of RNA Molecules that Bind and Thereby Increase the Fluorescence Intensity of a Fluorophore

The selection of RNA molecules that bind and enhance the fluorescence of a fluorophore is performed similar to the selection for DNA molecules described in Example 1.

Design of Candidate RNA Molecules

The pool of DNA molecules from Example 1 is transcribed into RNA molecules, using standard techniques (see, for example, U.S. Pat. No. 5,270,163; Ausubel et al., supra). Selection of RNA molecules that bind the fluorophore The pool of candidate RNA molecules is applied to a column containing the immobilized fluorophore; the column is washed, and the molecules that bind the fluorophore are eluted, as described in Example 1. To perform another round of the selection, the eluted RNA molecules are reverse transcribed to cDNA, and the cDNA is amplified using PCR. The PCR products are used as the templates for the next transcription to generate RNA molecules for the second round of selection.

Selection for RNA Molecules Which Bind and Increase the Fluorescence Intensity of the Fluorophore

For the in vitro selection of RNA molecules which also enhance the fluorescence of the fluorophore, the selected RNA molecules which bind the fluorophore are reverse transcribed to cDNA, and the cDNA is amplified using PCR. The PCR products are transcribed into RNA which is added to 96 well plates. The fluorophore is then added to the wells, and the plates are analyzed using a fluorescence imager.

To identify the RNA molecules in the wells with the highest fluorescence intensity, the RNA from these wells is reverse transcribed into cDNA, and the cDNA is inserted into a plasmid, transfected into bacteria, and sequenced as described in Example 1. The identified cDNA molecules are then transcribed into RNA molecules which are tested individually to determine which candidate RNA molecules were responsible for the fluorescence enhancement in the wells.

Alternatively, an in vivo selection may be performed to determine which candidate RNA molecules bind the fluorophore in vivo and enhance the fluorescence intensity of the fluorophore. For this method, the selected RNA molecules that bind the fluorophore are reversed transcribed into cDNA, and, if necessary, restriction sites for cloning are introduced using primers with these sites. The cDNA molecules are cloned into an appropriate plasmid or yeast artificial chromosome (YAC) under the control of a strong and easily inducible promoter, such as Lac, possibly at the 3′ end of a gene controlled by such a promoter. The cloning can be performed using standard molecular biology techniques, such as TOPO cloning (Shuman, J. Biol. Chem. 269:32678-32684, 1994). The plasmid or YAC is then transfected into eukaryotic or prokaryotic cells.

In the case of eukaryotes, such as yeast, the cells are grown to a density of 10⁶/ml and incubated in a buffer containing the fluorophore at a nanomolar to low micromolar or concentration, such as 100 nM to 50 μM. The fluorophore may then pass through the plasma membrane into the cytoplasm. The promoter is induced, allowing transcription of the induced gene to produce the candidate RNA molecule or an RNA molecule covalently linked to the candidate RNA molecule. The cells are sorted by FACS, and the cells with the highest fluorescence intensity are collected. Because even a million cells can be easily sorted by FACS, this method should detect cells having molecules that can enhance the fluorescence of the fluorophore, even if such molecules are rare. This in vivo selection may also be repeated several times to enrich the population for cells with high fluorescence.

For the bacterial selection system, the transfected bacteria are grown to produce single colonies. The colonies are picked manually or automatically using a robot and mixed so that 10 to 20 colonies are present per well in 96 well plates. The cells are grown to a density of 10⁸-10⁹/ml, the fluorophore is added to the wells, and the promoter is induced. The plates are screened for increased fluorescence intensity using a fluorescent imager, allowing up to 1,000,000 colonies to be imaged per day. If desired, immediately before imaging the plates, the buffer in the wells may be replaced by buffer that does not contain the fluorophore to decrease the background level of fluorescence due to unbound fluorophore in the buffer. The wells that have increased fluorescence over background are plated to produce single colonies which are added individually to a well, allowing the individual clones with enhanced fluoresce to be identified.

As a final screen in both the eukaryotic and prokaryotic selection methods, individual cells may be visualized with fluorescence microscopy to determine the cells with the most fluorescence enhancement. The plasmids or YACs are isolated from the cells, and the DNA encoding the candidate RNA molecules is sequenced using an oligonucleotide designed to bind the fixed primer sequence of the candidate molecules. Using these DNA sequences, the corresponding sequences of the RNA molecules that bind and increase the fluorescence intensity of the fluorophore are determined.

### EXAMPLE 3

### Selection of DNA or RNA Molecules That Bind and Thereby Decrease the Fluorescence Intensity of a Fluorophore

The selection of DNA or RNA molecules that bind and decrease the fluorescence of a fluorophore is performed similar to the selections for DNA and RNA molecules described in Examples 1 and 2.

Selection of DNA or RNA Molecules That Bind the Fluorophore

Candidate DNA or RNA molecules that bind a fluorophore may be selected as described in Examples 1 and 2.

Selection for DNA or RNA Molecules Which Bind and Decrease the Fluorescence Intensity of Tie Fluorophore

For the in vitro selection of DNA or RNA molecules which decrease the fluorescence of the fluorophore, the selected DNA or RNA molecules which bind the fluorophore are added to 96 well plates. The fluorophore is then added to the wells, and the plates are analyzed using a fluorescence imager. The DNA or RNA molecules in the wells with the lowest fluorescence intensity are identified using standard molecular biology techniques as described in Examples 1 and 2.

An in vivo selection may also be performed to determine which candidate RNA molecules bind the fluorophore in vivo and decrease the fluorescence intensity of the fluorophore. For this method, cDNA molecules encoding the selected RNA molecules are cloned into an appropriate plasmid or yeast artificial chromosome (YAC) under the control of an appropriate promoter as described in Example 2. The plasmid or YAC also contains an antibiotic resistance gene to allow selection of bacterial or eukaryotic (e.g., yeast) cells that have been transfected with the plasmid or YAC. The selected cells are then contacted with a fluorophore. The promoter is induced, allowing transcription of the induced gene to produce the candidate RNA molecule or an RNA molecule covalently linked to the candidate RNA molecule. The cells with the lowest fluorescence intensity may be identified using standard procedures, such as FACS analysis, fluorescent imaging, or fluorescence microscopy. The plasmids or YACs are isolated from the cells with the lowest fluorescence intensity and sequenced to determine the sequence of the encoded RNA molecules.

### EXAMPLE 4

### Visualization of a Specific Nucleic Acid Using the Selected Molecules That Enhance the Fluorescence of a Fluorophore

Expression of Fusion Molecules Having an Aptamer Linked to a Molecule of Interest

Standard gene fusion techniques may be used to generate fusion nucleic acid molecules that encode fusion RNA or DNA molecules including an aptamer covalently linked to a molecule of interest. For example, a fusion RNA molecule is generated by adding the DNA sequence that encodes an RNA aptamer to the 5′ end, 3′ end, or between the 5′ and 3′ ends of a DNA sequence encoding an RNA of interest. This recombinant DNA is transcribed in vitro or transfected, injected, or otherwise administered to cells for expression in vivo. It is also contemplated that a single-stranded fusion DNA molecule which contains a DNA aptamer and a DNA molecule of interest may be reversed transcribed by a recombinant RNA virus which has an RNA sequence corresponding to the sequence of the DNA fusion molecule.

Visualization of Fusion Molecules In Vitro

For visualization of these fusion RNA or DNA molecules in an in vitro sample, the fluorophore is added to the sample. If the aptamer binds the fluorophore with a dissociation constant of 100 nM or lower, only a few hundred nanomolar or less fluorophore is expected to be required to visualize the fusion molecules. The amount of fluorophore required depends on how much the fluorescence intensity increases when the fluorophore is bound to the fusion molecule, the length of time the fluorophore remains bound, and the number of fusion molecules present. If desired, fusion molecules that contain multiple copies of an aptamer or different aptamers may be used to increase the fluorescent signal. If necessary, the aptamers may be separated by a linker or added to different ends of the fusion molecule so that the fluorescence of one bound fluorophore is not quenched by a nearby bound fluorophore. The linker may consist of any nucleotide sequence and may be of any length. Preferably, the linker contains at least 5, 10, 25, or 50 nucleotides.

The fluorescence of the sample is detected and quantified, using standard techniques such as fluorimetry or fluorescence microscopy. Two or more fusion molecules that bind to fluorophores with different emission wavelengths may be visualized simultaneously by adding the fluorophores to the sample and analyzing the sample for emitted light at the various emission wavelengths. Using multiple fluorophores should enable the determination of fusion molecules that co-localize in the sample or in vivo. Additionally, if the emission wavelength of one bound fluorophore can induce the fluorescence of a second bound fluorophore, fluorescence resonance energy transfer between the fluorophores should be indicative of binding between the fusion molecules.

In addition to determining interactions between fusion nucleic acids, interactions between fusion nucleic acids and fusion proteins may be determined. To generate these fusion proteins, protein aptamers that bind an immobilized fluorophore or immobilized quencher may be selected using standard techniques, such as mRNA display (Publication No. WO 98/31700), ribosome display (Roberts, Curr. Opin. Chem. Biol. 3(3):268-73, 1999), or phage display for peptides (U.S. Pat. No. 5,821,047). The selected proteins that bind the fluorophore may be further tested to see if they also increase its fluorescence intensity in vivo. This selection may be performed using DNA that encodes the candidate proteins as described in Example 2 for DNA that encodes candidate RNA molecules. The DNA encoding the protein aptamers present in the cells with the highest fluorescence intensity may be isolated and sequenced to determine the sequence of the encoded protein aptamers. The DNA sequence encoding a protein aptamer may then be added to the DNA sequence encoding a protein of interest, and the resulting fusion protein may be expressed in an in vitro sample or a cell that also contains or expresses a fusion nucleic acid.

Visualization of Fusion Molecules In Vivo

For visualization of the fusion RNA or DNA in vivo, cells or embryos having DNA encoding the RNA fusion or a recombinant RNA virus expressing the DNA fusion are incubated with the fluorophore under conditions that allow the fluorophore to migrate through the cell wall and/or plasma membrane of the cells into the periplasm, and preferably, into the cytoplasm of the cell. Incubation of the cells in buffer containing a nanomolar to micromolar concentration of the fluorophore for a few minutes should allow the fluorophore to enter the cells. Although it is preferable to use a cell-permeable fluorophore, other fluorophore may be used by injecting them into the cells or embryos, using standard microinjection techniques. If desired, the buffer surrounding the cells may be changed prior to visualization of the cells to reduce the amount of fluorescence from the unbound fluorophore outside the cells.

The bound fluorophore in the cells is visualized using fluorescence microscopy or other standard techniques. FACS sorting may be used to sort the cells based on the fluorescence intensity and to quantify this intensity. This sorting should allow the identification of cells that have different amounts of the fusion molecule present, such as cells with different rates of transcription of the fusion molecule or different stability of these molecules.

For visualization of the fusion RNA that are present in a transgenic mammal or in a mammal, such as a human, that has undergone gene therapy, the fluorophore is administered to the mammal by any route that allows the fluorophore to migrate to the location of the fusion molecule. For example, the fluorophore may be intravenously or intravascularly administered to the mammal for migration for the fluorophore into cells in the bloodstream or other sites within the mammal accessible to the fluorophore. The fluorophore may also be injected into a site, such as a tissue, organ, or extracellular space, within a mammal in which cells having the fusion molecule are located. The administration of the fluorophore to a patient who has undergone gene therapy or to a transgenic animal may be used to determine which cells express an RNA molecule in which an aptamer is present.

### EXAMPLE 5

### Visualization of a Specific Nucleic Acid Using the Selected Molecules That Decrease the Fluorescence of a Fluorophore

As described in Example 4, fusion DNA molecules may be generated that contain a DNA sequence of interest linked to a DNA aptamer that binds and thereby decreases the fluorescence of a fluorophore. Similarly, fusion RNA molecules may be produced that contain a RNA sequence of interest linked to a RNA aptamer that binds and decreases the fluorescence of a fluorophore. A cell or in vitro sample containing one or more of these fusion DNA or RNA molecules may be contacted with the corresponding fluorophore. The presence of an area of decreased fluorescence intensity indicates the presence of a fusion DNA or RNA bound to the fluorophore.

Other Embodiments

From the foregoing description, it will be apparent that variations and modifications may be made to the invention described herein to adopt it to various usages and conditions. Such embodiments are also within the scope of the following claims.

All publications mentioned in this specification are herein incorporated by reference to the same extent as if each independent publication or patent application was specifically and individually indicated to be incorporated by reference.

What is claimed is:

1. A method of selecting an RNA molecule which binds a fluorophore, wherein said binding increases the fluorescence intensity of said fluorophore, said method comprising the steps of:
(a) providing a population of candidate RNA molecules;
(b) selecting said candidate RNA molecules which bind said fluorophore;
(c) contacting said candidate RNA molecules which bind said fluorophore with said fluorophore; and
(d) selecting said RNA molecules which, upon binding said fluorophore, increase its fluorescence intensity.

2. The method of claim 1, wherein said fluorophore is immobilized in step (b).

3. The method of claim 1, wherein step (c) comprises incubating cells with said fluorophore, wherein said cells contain one or more said candidate RNA molecules which bind said fluorophore.

4. The method of claim 3, wherein step (d) comprises sorting said cells based on fluorescence intensity and recovering the DNA coding sequences of said candidate RNA molecules or recovering said candidate RNA molecules from said sorted cells.

5. The method of claim 1, further comprising repeating steps (a) and (b).

6. The method of claim 1, further comprising repeating steps (c) and (d).

7. A method of selecting a DNA molecule which binds a fluorophore, wherein said binding increases the fluorescence intensity of said fluorophore, said method comprising the steps of:
(a) providing a population of candidate DNA molecules;
(b) selecting said candidate DNA molecules which bind said fluorophore;
(c) contacting said candidate DNA molecules which bind said fluorophore with said fluorophore; and
(d) selecting said DNA molecules which, upon binding said fluorophore, increase its fluorescence intensity.

8. The method of claim 7, wherein said fluorophore is immobilized in step (b).

9. The method of claim 7, wherein step (c) comprises incubating cells with said fluorophore, wherein said cells contain one or more said candidate DNA molecules which bind said fluorophore.

10. The method of claim 9, wherein step (d) comprises sorting said cells based on fluorescence intensity and recovering said candidate DNA molecules from said sorted cells.

11. The method of claim 7, further comprising repeating steps (a) and (b).

12. The method of claim 7, further comprising repeating steps (c) and (d).

13. A method of determining the presence, location, or quantity of an RNA of interest in a cell or an in vitro sample, said method comprising the steps of:
(a) expressing in said cell or said sample a fusion RNA comprising said RNA of interest covalently linked to an RNA aptamer;
(b) contacting said cell or said sample with a fluorophore, whereby said aptamer binds to said fluorophore and increases its fluorescence intensity; and
(c) visualizing or measuring the fluorescence of said fluorophore, thereby determining the presence, location, or quantity of said RNA of interest in said cell or said in vitro sample.

14. A method of determining the presence, location, or quantity of a DNA of interest in a cell or an in vitro sample, said method comprising the steps of:
(a) expressing in said cell or said sample a fusion DNA comprising said DNA of interest covalently linked to a DNA aptamer;
(b) contacting said cell or said sample with a fluorophore, whereby said aptamer binds to said fluorophore and increases its fluorescence intensity; and
(c) visualizing or measuring the fluorescence of said fluorophore, thereby determining the presence, location, or quantity of said DNA of interest in said cell or said in vitro sample.

15. A method of determining whether a compound is capable of modulating the transcription of an RNA of interest, said method comprising the steps of:
(a) expressing in a cell or an in vitro sample a fusion RNA comprising said RNA of interest covalently linked to an RNA aptamer;
(b) contacting said cell or said sample with either said compound and said fluorophore or with said fluorophore alone, whereby said aptamer binds to said fluorophore and increases its fluorescence intensity; and
(c) measuring said fluorescence intensity in the presence and absence of said compound, whereby said compound is determined to modulate said transcription if said compound effects a change in said fluorescence intensity.

16. The method of claim 15, wherein said compound is determined to be an inhibitor of said transcription if it decreases said fluorescence intensity.

17. The method of claim 15, wherein said compound is determined to be an inducer of said transcription if it increases said fluorescence intensity.

18. The method of claim 15, wherein said compound is a member of a library of at least 50 compounds, all of which are simultaneously contacted with said cell or said sample.

19. A method of determining whether a compound modulates the half-life of an RNA of interest, said method comprising the steps of:
(a) expressing in a cell or an in vitro sample a fusion RNA comprising said RNA of interest covalently linked to an RNA aptamer,
(b) contacting said cell or said sample with either said compound and a fluorophore or with a fluorophore alone, whereby said RNA aptamer binds to said fluorophore and increases its fluorescence intensity; and
(c) measuring said fluorescence intensity in the presence and absence of said compound, whereby said compound is determined to modulate said half-life if said compound effects a change in said fluorescence intensity.

20. The method of claim 19, wherein said compound is determined to decrease said half-life if it reduces said fluorescence intensity.

21. The method of claim 19, wherein said compound is determined to increase said half-life if it increases said fluorescence intensity.

22. The method of claim 19, wherein said compound is a member of a library of at least 50 compounds, all of which are simultaneously contacted with said cell or said sample.

23. The method of claim 19, wherein said compound degrades, induces the degradation, or suppresses the degradation of said RNA of interest.

24. A method of determining whether an antisense nucleic acid binds a target RNA in a cell or an in vitro sample, said method comprising the steps of:
(a) expressing in said cell or said sample a fusion RNA comprising said target RNA covalently linked to an RNA aptamer;
(b) contacting said cell or said sample with either said antisense nucleic acid and a fluorophore or with a fluorophore alone, whereby said RNA aptamer binds to said fluorophore and increases its fluorescence intensity; and
(c) measuring said fluorescence intensity in the presence and absence of said antisense nucleic acid, whereby said antisense nucleic acid is determined to bind said target RNA if said antisense nucleic acid decreases said fluorescence intensity.

25. A method of determining whether an antisense nucleic acid binds a target RNA in a cell or an in vitro sample, said method comprising the steps of:
(a) expressing in said cell or said sample a fusion RNA comprising said target RNA covalently linked to an RNA aptamer which binds a first fluorophore;
(b) contacting said cell or said sample with either said first fluorophore and said antisense nucleic acid covalently linked to a second fluorophore or with said first fluorophore alone, whereby said RNA aptamer binds to said first fluorophore and increases its fluorescence intensity; and wherein the emission wavelength of said first fluorophore is different from that of said second fluorophore, and the emission wavelength of said second fluorophore induces the fluorescence of said first fluorophore; and
(c) measuring said fluorescence intensity of said first fluorophore in the presence and absence of said antisense nucleic acid; whereby said antisense nucleic acid is determined to bind said target RNA if said antisense nucleic acid increases said fluorescence -intensity of said first fluorophore.

26. A method of determining whether an antisense nucleic acid binds a target RNA in a cell or an in vitro sample, said method comprising the steps of:
(a) expressing in said cell or said sample a fusion RNA comprising said target RNA covalently linked to an RNA aptamer which binds a fluorophore;
(b) contacting said cell or said sample with either said fluorophore and said antisense nucleic acid covalently linked to a quencher or with said fluorophore alone, whereby said RNA aptamer binds to said fluorophore and increases its fluorescence intensity; and wherein the fluorescence intensity of said fluorophore is decreased by said quencher; and
(e) measuring said fluorescence intensity of said fluorophore in the presence and absence of said antisense nucleic acid; whereby said antisense nucleic acid is determined to bind said target RNA if said antisense nucleic acid decreases said fluorescence intensity.

27. A method of determining whether an antisense nucleic acid binds a target RNA in a cell or an in vitro sample, said method comprising the steps of:
(a) expressing in said cell or said sample a fusion RNA comprising said target RNA covalently linked to an RNA aptamer which binds a first fluorophore;
(b) contacting said cell or said sample with either said first fluorophore, said antisense nucleic acid covalently linked to a nucleic acid aptamer which binds a second fluorophore, and said second fluorophore or with said first fluorophore alone, whereby said RNA aptamer binds to said first fluorophore and increases its fluorescence intensity, and said nucleic acid aptamer binds to said second fluorophore and increases its fluorescence intensity; and wherein the emission wavelength of said first fluorophore is different from that of said second fluorophore, and the emission wavelength of said second fluorophore induces the fluorescence of said first fluorophore; and
(c) measuring said fluorescence intensity of said first fluorophore in the presence and absence of said antisense nucleic acid, whereby said antisense nucleic acid is determined to bind said target RNA if said antisense nucleic acid increases said fluorescence intensity of first fluorophore.

28. A method of determining whether an antisense nucleic acid binds a target RNA in a cell or an in vitro sample, said method comprising the steps of:
(a) expressing in said cell or said sample a fusion RNA comprising said target RNA covalently linked to an RNA aptamer which binds a fluorophore;
(b) contacting said cell or said sample with said fluorophore, said antisense nucleic acid covalently linked to a nucleic acid aptamer which binds a quencher, and said quencher, or with said fluorophore alone, whereby said RNA aptamer binds to said fluorophore and increases its fluorescence intensity, and said nucleic acid aptamer binds to said quencher; and wherein the fluorescence intensity of said fluorophore is decreased by said quencher; and
(c) measuring said fluorescence intensity of said fluorophore in the presence and absence of said antisense nucleic acid, whereby said antisense nucleic acid is determined to bind said target RNA if said antisense nucleic acid decreases said fluorescence intensity.

29. A method of determining whether a first RNA is co-localized with a second RNA of interest in a cell or an in vitro sample, said method comprising the steps of:
(a) expressing in said cell or said sample a first fusion RNA comprising said first RNA covalently linked to a first RNA aptamer which binds a first fluorophore;
(b) expressing in said cell or said sample a second fusion RNA comprising said second RNA of interest covalently linked to a second RNA aptamer which binds a second fluorophore, wherein the emission wavelength of said second fluorophore is different from that of said first fluorophore;
(c) contacting said cell or said sample with said first fluorophore and said second fluorophore, whereby said first fluorophore binds to said first RNA aptamer and said second fluorophore binds to said second RNA aptamer, and whereby said binding increases the fluorescence intensity of said first fluorophore and said second fluorophore; and
(d) visualizing the fluorescence of said first fluorophore and said second fluorophore, whereby said first RNA of interest is determined to co-localize with said second RNA of interest if said fluorescence of said first fluorophore is detected proximal to the fluorescence of said second fluorophore.

30. A method of determining whether an RNA of interest is co-localized with a protein of interest in a cell or an in vitro sample, said method comprising the steps of:
(a) expressing in said cell or said sample a fusion RNA comprising said RNA of interest covalently linked to an RNA aptamer which binds a first fluorophore;
(b) expressing in said cell or said sample a fusion protein comprising said protein of interest covalently linked to a detectable protein;
(c) contacting said cell or said sample with said first fluorophore, whereby said RNA aptamer binds to said first fluorophore and increases its fluorescence intensity; and
(d) visualizing the fluorescence of said first fluorophore and determining the localization of said detectable protein, whereby said first RNA of interest is determined to co-localize with said protein of interest if said fluorescence of said first fluorophore is detected proximal to said detectable protein.

31. The method of claim 30, wherein said detectable protein has intrinsic fluorescence or luminescence and wherein the localization of said detectable protein is determined by visualizing its fluorescence or luminescence.

32. The method of claim 31, wherein said detectable protein is a green fluorescent protein.

33. The method of claim 30, further comprising contacting said cell or said sample with a second fluorophore, whereby said detectable protein binds to said second fluorophore and increases its fluorescence intensity; and wherein the localization of said detectable protein is determined by visualizing the fluorescence of said second fluorophore.

34. A method of determining whether a first RNA of interest interacts with a second RNA of interest in a cell or an in vitro sample, said method comprising the steps of:
(a) expressing in said cell or said sample a first fusion RNA comprising said first RNA of interest covalently linked to a first RNA aptamer which binds a first fluorophore;
(b) expressing in said cell or said sample a second fusion RNA comprising said second RNA of interest covalently linked to a second RNA aptamer which binds a second fluorophore, wherein the emission wavelength of said first fluorophore is different from that of said second fluorophore, and wherein the emission wavelength of said first fluorophore induces the fluorescence of said second fluorophore;
(c) contacting said cell or said sample with either said first fluorophore and said second fluorophore or with said second fluorophore alone, whereby said first fluorophore binds to said first RNA aptamer and said second fluorophore binds to said second RNA aptamer, and whereby said binding increases the fluorescence intensity of said first fluorophore and said second fluorophore; and
(d) measuring said fluorescence intensity of said second fluorophore in the presence and absence of said first fluorophore, whereby said first RNA of interest is determined to interact with said second RNA of interest if said first fluorophore induces said fluorescence intensity of said second fluorophore.

35. A method of determining whether an RNA of interest interacts with a protein of interest in a cell or an in vitro sample, said method comprising the steps of:
(a) expressing in said cell or said sample a fusion RNA comprising said RNA of interest covalently linked to a RNA aptamer which binds a first fluorophore;
(b) expressing in said cell or said sample a fusion protein comprising said protein of interest covalently linked to detectable protein which binds a second fluorophore, wherein the emission wavelength of said first fluorophore is different from that of said second fluorophore, and wherein the emission wavelength of said first fluorophore induces the fluorescence of said second fluorophore or the emission wavelength of said second fluorophore induces the fluorescence of said first fluorophore;
(c) contacting said cell or said sample with either said first fluorophore and said second fluorophore, said first fluorophore alone, or said second fluorophore alone, whereby said RNA aptamer binds to said first fluorophore and increases its fluorescence intensity, and whereby said detectable protein binds to said second fluorophore and increases its fluorescence intensity; and
(d) measuring said fluorescence intensity of said first fluorophore in the presence and absence of said second fluorophore or measuring said fluorescence intensity of said second fluorophore in the presence and absence of said first fluorophore, whereby said RNA of interest is determined to interact with said protein of interest if fluorescence resonance energy transfer occurs between said first fluorophore and said second fluorophore.

36. A method of determining whether an RNA of interest interacts with a protein of interest in a cell or an in vitro sample, said method comprising the steps of:
(a) expressing in said cell or said sample a fusion RNA comprising said RNA of interest covalently linked to a RNA aptamer which binds a fluorophore;
(b) expressing in said cell or said sample a fusion protein comprising said protein of interest covalently linked to detectable protein with intrinsic fluorescence, wherein the emission wavelength of said fluorophore is different from that of said detectable protein, and wherein the emission wavelength of said fluorophore induces the fluorescence of said detectable protein or wherein the emission wavelength of said detectable protein induces the fluorescence of said fluorophore;
(c) contacting said cell or said sample with said fluorophore, whereby said RNA aptamer binds to said fluorophore and increases its fluorescence intensity; and
(d) measuring said fluorescence intensity of said fluorophore in the presence and absence of said detectable protein or measuring said fluorescence intensity of said detectable protein in the presence and absence of said fluorophore, whereby said RNA of interest is determined to interact with said protein of interest if fluorescence resonance energy transfer occurs between said fluorophore and said detectable protein.

37. The method of claim 13-15, 19, 24-30, or 34-36, wherein said cell is a prokaryotic cell.

38. The method of claim 37, wherein said cell is a gram-negative or gram-positive bacterial cell.

39. The method of claim 13-15, 19, 24-30, or 34-36, wherein said cell is a eukaryotic cell.

40. The method of claim 39, wherein said cell is a yeast, Caenorhabditis, Xenopus, Drosophila, zebrafish, squid, plant, mammalian, or human cell.

41. The method of claim 13-15, 19, 24-30, or 34-36, wherein said cell is an embryonic cell.

42. The method of claim 13-15, 19, 24-30, or 34-36, wherein said cell is in a mammal.

43. The method of claim 13-15, 19, 24-30, or 34-36, wherein said contacting comprises incubating said cell or said sample with said fluorophore.

44. The method of claim 13-15, 19, 24-30, or 34-36, wherein said contacting comprises injecting said fluorophore into said cell.

45. A population of nucleic acids having two loops separated by a base-paired helix, wherein at least one nucleic acid has a non-naturally-occurring polynucleotide sequence.

46. A population of nucleic acids having a loop flanked by a base-paired helix on one side and a hairpin on the other side, wherein at least one nucleic acid has a non-naturally-occurring polynucleotide sequence.

47. A population of nucleic acids having a pseudoknot structure, wherein at least one nucleic acid has a non-naturally-occurring polynucleotide sequence.
